(19) United States
(12) Patent Application Publication
(10) Pub. No.: US 2024/0225897 A1
(43) Pub. Date: Jul. 11, 2024

(54) APPARATUS FOR CUTTING A HUMAN OR ANIMAL TISSUE COMPRISING AN OPTICAL COUPLER

(71) Applicant: KERANOVA, SAINT ETIENNE (FR)

(72) Inventors: Sylvie NADOLNY, MIONS (FR); Emmanuel BAUBEAU, SAINT ETIENNE (FR)

(21) Appl. No.: 18/609,999

(22) Filed: Mar. 19, 2024

Related U.S. Application Data
(63) Continuation of application No. 16/964,234, filed on Jul. 23, 2020.

(30) Foreign Application Priority Data
Jan. 25, 2018 (FR) 1850577

(51) Int. Cl. A61F 9/008

(52) U.S. Cl. CPC A61F 9/0084; A61F 2009/00844; A61F 2009/0087; A61F 2009/00872; A61F 2009/00897

(57) ABSTRACT

The present invention concerns a cutting apparatus including a femtosecond laser, a shaping system downstream from the femtosecond laser, for forming a phase-modulated laser beam, an optical scanner downstream from the shaping system, and optical focusing system downstream from the optical scanner, a control unit for controlling the shaping system, the optical scanner and the optical focusing system, characterized in that the apparatus further comprises an optical coupler between the femtosecond laser and the shaping system, the optical coupler including a photonic crystal optical fiber for filtering the phase-modulated laser beam coming from the shaping system.

### CROSS-REFERENCE TO RELATED APPLICATIONS

This application is a continuation of U.S. patent application Ser. No. 16/964,234, filed on Jul. 23, 2020, which is a National Phase Entry under 35 U.S.C. § 371 of International Application No. PCT/EP2019/051872 filed on Jan. 25, 2019, which claims benefit of priority from French Patent Application No. 1850577 filed Jan. 25, 2018, the contents of each of which are hereby incorporated by reference in their entirety.

### TECHNICAL FIELD

The present invention relates to the technical field of the treatment of ocular pathologies performed by using a femtosecond laser, and more particularly that of the ophthalmological surgery in particular for applications to cut corneas or crystalline lenses.

The invention relates to a device for cutting a human or an animal tissue, such as a cornea, or a crystalline lens, by means of a femtosecond laser.

By femtosecond laser is meant a light source able to emit a LASER beam in the form of ultra-short pulses, the duration of which is comprised between 1 femtosecond and 100 picoseconds, preferably between 1 and 1000 femtoseconds, in particular on the order of one hundred femtoseconds.

### PRIOR ART

It has already been proposed to perform surgical operations on the eye by means of a femtosecond laser, such as operations of cutting corneas or crystalline lenses.

Document FR 3 049 847 describes an apparatus for cutting a human or an animal tissue, such as a cornea, or a crystalline lens. This apparatus includes:
a femtosecond laser to generate a LASER beam,
a shaping system positioned on the path of said beam, to modulate the phase of the wave front of the LASER beam so as to obtain a phase-modulated LASER beam based on a modulation instruction calculated to distribute the energy of the LASER beam into at least two impact points forming a pattern in its focal plane corresponding to a cutting plane,
an optical scanner disposed downstream of the shaping system to move the pattern in the cutting plane into a plurality of positions along a direction of movement,
an optical assembly including reflective mirrors and lenses between the shaping system and the optical scanner for the transmission of the modulated LASER beam towards the scanner,
an optical focusing system to focus the LASER beam in a cutting plane.

The use of a shaping system allows reducing the biological tissue cutting time by generating several impact points simultaneously.

Furthermore, the use of the shaping system allows obtaining substantially equal impact points (the shape, the position and the diameter of each point being dynamically monitored by a phase mask calculated and displayed on the shaping system).

Thus, the gas bubbles—generated by the impact points and—which dilacerate the cut biological tissues are of approximately equal sizes.

This allows improving the quality of the result obtained, with a homogeneous cutting plane, in which the residual tissue bridges (between adjacent impact points) have all substantially the same size. This homogeneity in the size of the tissue bridges allows dissection by the practitioner of an acceptable quality with regard to the importance of the quality of the surface condition of the cut tissue when it is for example a cornea.

However, to facilitate the operation of dissection by the practitioner, it is preferable to reduce the size of the residual tissue bridges between adjacent impact points.

As this size of the tissue bridges depends on the homogeneity of the different impact points, an aim of the present invention is to propose a technical solution that allows improving the homogeneity of the distribution of energy between the different impact points generated simultaneously thanks to the shaping system.

Another aim of the present invention is to propose a technical solution that allows improving the apparatus described in document FR 3 049 847 in order to reduce the size of the residual tissue bridges between adjacent impact points.

Yet another aim of the present invention is to improve the security of the apparatus described in document FR 3 049 847 by incorporating therein a security member that allows interrupting the transmission of the laser beam up to the tissue to be treated if said laser beam becomes offset (for example in the event of an impact on the apparatus).

### DISCLOSURE OF THE INVENTION

To this end, the invention proposes an apparatus for cutting a human or an animal tissue, such as a cornea, or a crystalline lens, said apparatus including:
a femtosecond laser to emit an initial LASER beam in the form of pulses,
a shaping system—such as a Spatial Light Modulator (SLM)—positioned downstream of the femtosecond laser, to transform the initial LASER beam into phase-modulated LASER beam, the shaping system being able to modulate the phase of the wave front of the initial LASER beam according to a modulation instruction calculated to distribute the energy of the LASER beam into at least two impact points forming a pattern in a focusing plane,
an optical scanner, positioned downstream of the shaping system, to move the pattern along a predefined movement path in the focusing plane,
an optical focusing system, positioned downstream of the optical scanner, to move the focusing plane of the modulated LASER beam in a desired cutting plane of the tissue,
a control unit that allows piloting the shaping system, the optical scanner and the optical focusing system,
remarkable in that the apparatus further comprises an optical coupler between the femtosecond laser and the shaping system, the optical coupler including a photonic-crystal optical fiber for the filtering of the LASER beam derived from the femtosecond laser.

Within the context of the present invention, it is meant by “impact point” an area of the LASER beam comprised in its focal plane in which the intensity of said LASER beam is sufficient to generate a gas bubble in a tissue.

Within the context of the present invention, it is meant by “adjacent impact points” two impact points disposed facing one another and not separated by another impact point. It is meant by “neighboring impact points” two points in a group of adjacent points between which the distance is minimum.

Within the context of the present invention, it is meant by “pattern” a plurality of LASER impact points generated simultaneously in a focusing plane of a shaped—that is to say phase-modulated—LASER beam to distribute its energy into several distinct spots in the focusing plane corresponding to the cutting plane of the device.

Thus, the invention makes it possible to modify the intensity profile of the LASER beam in the cutting plane, so as to be able to improve the cutting quality or speed according to the chosen profile. This modification of intensity profile is obtained by modulation of the phase of the LASER beam.

The optical phase modulation is performed by means of a phase mask. The energy of the incident LASER beam is preserved after modulation, and the shaping of the beam is performed by acting on its wave front. The phase of an electromagnetic wave represents the instantaneous situation of the amplitude of an electromagnetic wave. The phase depends both on time and space. In the case of the spatial shaping of a LASER beam, only the variations in the space of the phase are considered.

The wave front is defined as the surface of the points of a beam having an equivalent phase (i.e. the surface made up of points whose travel times from the source having emitted the beam are equal). The modification of the spatial phase of a beam therefore involves the modification of its wave front.

This technique allows performing the cutting operation in a faster and more effective way because it implements several LASER spots each carrying out a cutout and according to a monitored profile.

The fact of positioning the optical coupler including the photonic-crystal optical fiber between the femtosecond laser and the shaping system (rather than between the shaping system and the optical scanner) allows voiding any disturbance in the shaping of the laser beam carried out by the shaping system. Indeed, the introduction of an optical coupler including a photonic-crystal fiber between the shaping system and the optical scanner would induce a filtering of the modulated laser beam (coming from the shaping system) that tends to degrade its shaping and to decrease its power.

Preferred but non-limiting aspects of the cutting apparatus are as follows:
The fiber can be a hollow-core photonic-crystal fiber, said fiber including a hollow core, and at least one sheath surrounding the hollow core;
the optical coupler can further comprise:
   a first connection cell for linking the optical coupler to the shaping system on the one hand, and
   a second connection cell for linking the optical coupler to the optical scanner on the other hand;
each connection cell can be sealingly mounted at a respective end of the photonic-crystal fiber;
each connection cell can comprise:
   an outer shell,
   a transmission channel housed in the shell, the transmission channel allowing the passage of the LASER beam inside the shell,
   a window transparent to the LASER radiation at one end of the transmission channel, the window being intended to face the femtosecond laser or the shaping system;
the apparatus can further comprise at least one vacuum pump, each connection cell comprising at least one connection terminal opening out towards the outside of the shell and being intended to be linked to the vacuum pump;
the control unit can comprise means able to pilot the activation of the vacuum pump to suck the gases contained in the hollow core of the photonic-crystal optical fiber.

### BRIEF DESCRIPTION OF THE DRAWINGS

Other characteristics and advantages of the invention will emerge clearly from the description which is made below, for indication and without limitation, with reference to the appended figures, wherein:

FIG. 1 is a schematic representation of an assembly including the cutting apparatus according to the invention;

FIG. 2 illustrates a distribution of intensity of a LASER beam in its focal plane;

FIG. 3 illustrates an example of an optical coupler of the cutting apparatus illustrated in FIG. 1;

FIG. 4 illustrates a path of movement of a cutting pattern;

FIG. 5 illustrates cutting planes of a volume of tissue to be destroyed;

FIG. 6 illustrates a therapy apparatus including an articulated arm.

### DETAILED DISCLOSURE OF THE INVENTION

The invention relates to an apparatus for cutting a human or an animal tissue by means of a femtosecond laser. In the following description, the invention will be described, by way of example, for the cutting of a cornea of a human or an animal eye.

### 1. Cutting Apparatus

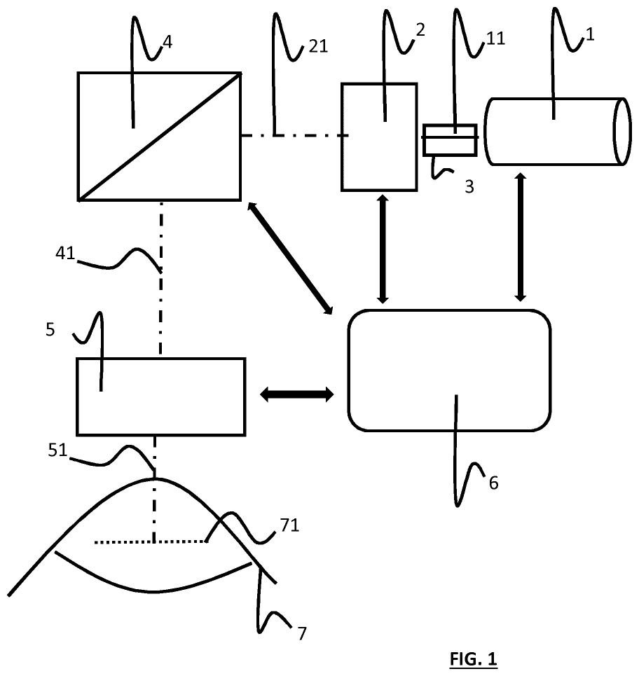

Referring to FIG. 1, one embodiment of the cutting apparatus according to the invention is illustrated. It can be disposed upstream of a target to be treated 7. The target 7 is for example a human or an animal tissue to be cut such as a cornea or a crystalline lens.

The cutting apparatus comprises:
a femtosecond laser 1,
a shaping system 2 positioned downstream of the femtosecond laser 1,
an optical coupler 3 between the femtosecond laser 1 and the shaping system 2,
an optical scanner 4 downstream of the shaping system 2,
an optical focusing system 5 downstream of the optical scanner 4,
a control unit 6 that allows piloting the femtosecond laser 1, the shaping system 2, the optical scanner 4 and the optical focusing system 5.

The femtosecond laser 1 is able to emit an initial LASER beam in the form of pulses. For example, the laser 1 emits a light of a wavelength of 1030 nm, in the form of pulses of 400 femtoseconds. The laser 1 has a power comprised between 2 and 20 W and preferably on the order of 8 W and a frequency comprised between 100 and 500 kHz.

The optical coupler 3 allows transmitting the LASER beam 11 derived from the femtosecond laser 1 towards the shaping system 2.

The shaping system 2 extends over the path of the initial LASER beam 11 derived from the femtosecond laser 1. It allows transforming the initial LASER beam 11 into a modulated LASER beam 21. More specifically, the shaping system 2 allows modulating the phase of the LASER beam 11 to distribute the energy of the LASER beam into a plurality of impact points in its focal plane, this plurality of impact points defining a pattern 8.

The optical scanner 4 allows orienting the modulated LASER beam 21 to move the pattern 8 along a movement path predefined by the user in a focusing plane 71.

The optical focusing system 5 allows moving the focusing plane 71—corresponding to the cutting plane—of the deflected LASER beam 41 coming from the optical scanner 4.

Thus:
the optical coupler 3 allows propagating the LASER beam 11 between the femtosecond laser and the shaping system 2,
the shaping system 2 allows simultaneously generating several impact points 81 defining a pattern 8,
the optical scanner 4 allows moving this pattern 8 in the focusing plane 71, and
the optical focusing system 5 allows moving the focusing plane 71 in depth so as to generate cutouts in successive planes defining a volume.

The various elements constituting the cutting apparatus will now be described in more detail with reference to the figures.

### 2. Elements of the Cutting Apparatus

### 2.1. Shaping System

The spatial shaping system 2 allows varying the wave surface of the initial LASER beam 11 to obtain impact points 8 separated from each other in the focusing plane 71.

More specifically, the shaping system 2 allows modulating the phase of the initial LASER beam 11 derived from the femtosecond laser 1 to form intensity peaks in the focusing plane 71, each intensity peak producing a respective impact point in the focal plane corresponding to the cutting plane. The shaping system 2 is, according to the illustrated embodiment, a liquid-crystal Spatial Light Modulator, known by the acronym SLM.

The SLM allows modulating the final energy distribution of the LASER beam, in particular in the focusing plane 71 corresponding to the cutting plane of the tissue 7. More specifically, the SLM is adapted to modify the spatial profile of the wave front of the primary LASER beam 11 derived from the femtosecond laser 1 to distribute the energy of the LASER beam into different focusing spots in the focusing plane 71.

The phase-modulation of the wave front can be seen as a two-dimensional interference phenomenon. Each portion of the initial LASER beam 11 derived from the source 1 is delayed or advanced relative to the initial wave front so that each of these portions is redirected so as to produce constructive interference in N distinct points in the focal plane of a lens. This redistribution of energy into a plurality of impact points 81 takes place only in a single plane (i.e. the focusing plane 71) and not along the propagation path of the modulated LASER beam. Thus, the observation of the modulated LASER beam before or after the focusing plane does not allow identifying a redistribution of the energy into a plurality of distinct impact points 81, because of this phenomenon which can be assimilated to constructive interferences (which take place only in one plane and not throughout the propagation as in the case of the splitting of an initial LASER beam into a plurality of secondary LASER beams).

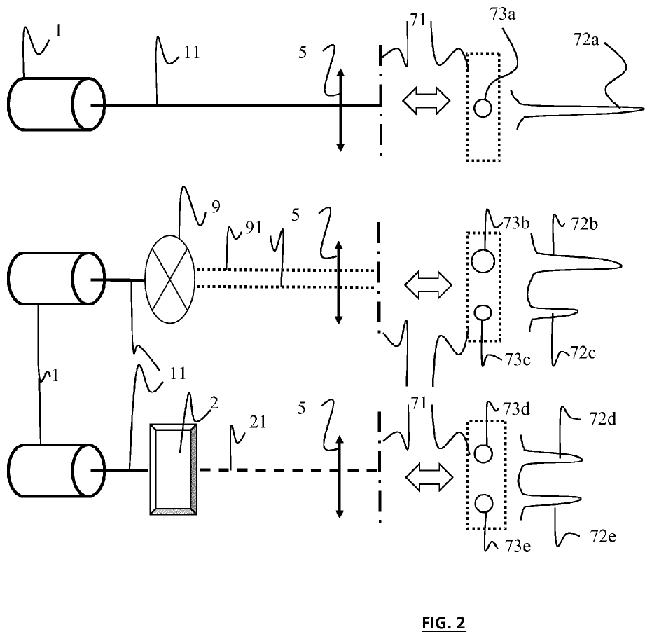

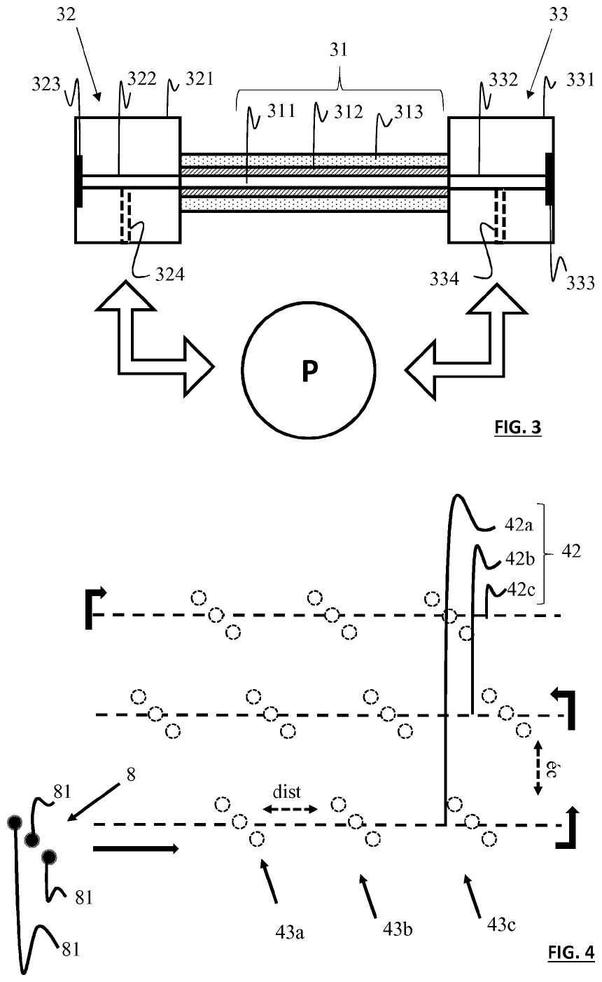

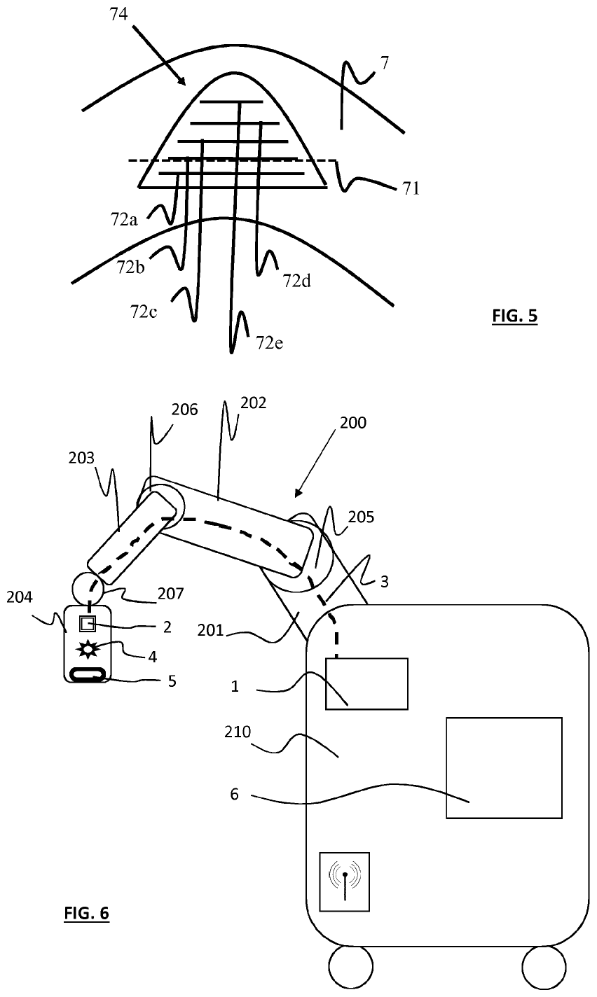

To better understand this phenomenon of phase-modulation of the wave front, intensity profiles 72a-72e obtained for three examples of distinct optical assemblies were schematically illustrated in FIG. 2. As represented in FIG. 2, an initial LASER beam 11 emitted by a laser source 1 produces a Gaussian-shaped intensity peak 72a at an impact point 73a in a focusing plane 71. The insertion of a beam splitter 9 between the source 1 and the focusing plane 71 induces the generation of a plurality of secondary LASER beams 91, each secondary LASER beam 91 producing a respective impact point 73b, 73c in the focusing plane 71 of the secondary LASER beams 91. Finally, the insertion between the source 1 and the focusing plane 71 of an SLM 2 programmed using a phase mask forming a modulation instruction induces the modulation of the phase of the wave front of the initial LASER beam 11 derived from the source 1. The LASER beam 21 whose wave front phase has been modulated allows inducing the production of several peaks of intensity 73d, 73e spatially separated in the focusing plane 71, each peak 72d, 72e corresponding to a respective impact point 73d, 73e performing a cutout. The wave front phase modulation technique allows generating in the target tissue several simultaneous gas bubbles without multiplication of the initial LASER beam 11 produced by the femtosecond laser 1.

The SLM is a device composed of a layer of liquid crystals with monitored orientation making it possible to dynamically shape the wave front, and therefore the phase of the LASER beam. The layer of liquid crystals of an SLM is organized like a grid (or matrix) of pixels. The optical thickness of each pixel is electrically monitored by orientation of the liquid-crystal molecules belonging to the surface corresponding to the pixel. The SLM exploits the principle of anisotropy of the liquid crystals, that is to say the modification of the index of liquid crystals, according to their spatial orientation. The orientation of the liquid crystals can be achieved using an electric field. Thus, the modification of the index of the liquid crystals modifies the wave front of the LASER beam.

In a known manner, the SLM implements a phase mask, that is to say a map determining how the phase of the beam must be modified to obtain a distribution of amplitude given in its focusing plane 71. The phase mask is a two-dimensional image, each point of which is associated with a respective pixel of the SLM. This phase mask allows piloting the index of each liquid crystal of the SLM by converting the value associated with each point of the mask—represented in gray levels comprised between 0 and 255 (therefore from black to white)—into a control value—represented in a phase comprised between 0 and 2π. Thus, the phase mask is a modulation instruction displayed on the SLM to cause in reflection an uneven spatial phase-shift of the LASER beam illuminating the SLM. Of course, those skilled in the art will appreciate that the gray level range may vary according to the SLM model used. For example in some cases, the gray level range can be comprised between 0 and 220. The phase mask is generally calculated by an iterative algorithm based on the Fourier transform, or on various optimization algorithms, such as genetic algorithms, or the simulated annealing. Different phase masks can be applied to the SLMs depending on the number and position of the desired impact points in the focal plane of the LASER beam. In all cases, those skilled in the art know how to calculate a value at each point of the phase mask to distribute the energy of the LASER beam into different focusing spots in the focal plane.

The SLM therefore allows, from a Gaussian LASER beam generating a single impact point and by means of the phase mask, distributing its energy by phase-modulation so as to simultaneously generate several impact points in its focusing plane from a single LASER beam shaped by phase-modulation (a single beam upstream and downstream of the SLM).

In addition to a reduction of the cornea cutting time, the technique of modulation of the LASER beam phase allows for other improvements, such as better surface quality after cutting or a reduction in the endothelial mortality. The different impact points of the pattern can, for example, be evenly spaced on the two dimensions of the focal plane of the LASER beam, so as to form a grid of LASER spots.

Thus, the shaping system 2 allows performing a surgical cutting operation quickly and effectively. The SLM allows dynamically shaping the wave front of the LASER beam since it is digitally parameterizable. This modulation allows the shaping of the LASER beam in a dynamic and reconfigurable way.

The SLM can be configured to shape the wave front of the LASER beam in any other way. For example, each impact point can have any geometric shape, other than circular (for example elliptical, etc.). This can have some advantages depending on the considered application, such as an increase in the speed and/or in the quality of the cutout.

### 2.2. Optical Coupler

The optical coupler 3 allows the transmission of the LASER beam 11 between the femtosecond laser 1 and the shaping system 2.

Referring to FIG. 3, the optical coupler 3 advantageously comprises an optical fiber 31. This allows the optical coupler 3 to constitute an “optical fuse”. Indeed, if the direction of the LASER beam 11 (i.e. its viewing point) is suddenly modified—for example in the event of an impact on the cutting device—then the LASER beam 11 no longer penetrates the fiber, which limits the risks of error when treating a patient. This is not possible with an optical assembly including reflective mirrors and lenses for the transmission of the LASER beam derived from the femtosecond laser.

Advantageously, the optical fiber 31 can be a photonic-crystal fiber. A Photonic-Crystal Fiber or “PCF” are waveguides formed of a periodic network in two dimensions of inclusions which extend over the entire length of the fiber. The transmission of a LASER beam through such a fiber is based on the properties of the photonic-crystals. Thanks to their structures, these fibers ensure the confinement of electromagnetic waves in the core of the fiber. These photonic-crystal fibers offer a wide variety of possibilities for the guidance by adjusting their optogeometric parameters such as for example the diameter of the inclusions, the distribution of the inclusions, the periodicity (not between two inclusions), the number of layers, the index of the used materials.

Preferably, the optical fiber 31 is a hollow-core photonic-crystal fiber. A hollow-core photonic-crystal fiber is an optical fiber which guides light the essentially inside a hollow region (the core of the fiber), so that only a minor part of the optical power propagates in the solid fiber material (typically silica). According to the standard physical mechanism for guiding the light into a fiber, this should not be possible: normally, the refractive index of the fiber core should be higher than that of the surrounding sheathing material, and there is no means for obtaining a refractive index of glass below that of air or vacuum, at least in the optical region. However, a different guide mechanism can be used, based on a photonic band gap, as can be done in a photonic-crystal fiber. Such fibers are also called photonic band gap fibers. The appeals for the hollow-core photonic-crystal fibers are mainly that the primary guidance in the hollow region minimizes the non-linear effects of the LASER beam 11 and allows a high damage threshold.

By way of example, document FR 3 006 774 describes a waveguide in the form of a hollow-core photonic-crystal fiber comprising a sheath, the absence of capillary in the central part forming the hollow core. The use of a hollow-core photonic-crystal fiber allows filtering the LASER beam 11 derived from the femtosecond laser 1 in order to facilitate its shaping by the shaping system 2. More specifically, the use of a hollow-core photonic-crystal fiber allows limiting the divergence of the LASER beam 11 (i.e. spread profile) by improving its directivity (which makes the LASER beam 11 cleaner by limiting the spreading of its profile). Indeed, a hollow-core photonic-crystal fiber allows confining the light more effectively than a conventional solid-core fiber. The hollow-core photonic-crystal fiber comprises:
a hollow core 311,
an inner sheath 312 based on silica surrounding the hollow core, the inner sheath having a refractive index n1<nc, where nc is the effective refractive index of the hollow core,
an outer sheath 313 surrounding the inner sheath 312.

Advantageously, the hollow region 311 of the hollow-core photonic-crystal fiber can be placed under vacuum to limit the propagation losses of the LASER beam 11 derived from the femtosecond laser 1. As a variant, a gas can be injected into the hollow region to exploit the high optical intensity in the fiber—for example for a high harmonic generation of the LASER beam 11 derived from the femtosecond laser 1. For this purpose, the optical coupler 3 comprises first and second connection cells 32, 33 sealingly mounted at each end of the hollow-core photonic-crystal fiber.

Each connection cell 32, 33 comprises:
an outer shell 321, 331,
a transmission channel 322, 332 housed in the shell 321, 331, the transmission channel 322, 332 allowing the passage of the LASER beam 11 inside the shell 321, 331,
a window 323, 333 transparent to the LASER radiation at one end of the transmission channel 322, 332 for the entrance (or the exit) of the LASER beam 11,
a connector (not represented) at the other end of the transmission channel, the connector being sealingly connected to one end of the optical fiber 31,
a connection terminal 324, 334 opening out towards the outside of the shell 321, 331 and being intended to be connected to a vacuum pump P.

The activation of the vacuum pump P allows placing the hollow core 311 of the optical fiber 31 under vacuum by pumping at the connection cells 32, 33 located at both ends of the optical fiber 31. The fact of carrying out a vacuum pumping at each end of the optical fiber 31 makes it easier to place under vacuum the hollow core over the entire length of the optical fiber 31.

### 2.3. Optical Scanner

The optical scanner 4 allows deflecting the phase-modulated LASER beam 21 so as to move the pattern 8 into a plurality of positions 43a-43c in the focusing plane 71 corresponding to the cutting plane.

The optical scanner 4 comprises:
an entrance orifice linked to the optical coupler 3 to receive the phase-modulated LASER beam 21 coming from the shaping unit 2,
one (or more) optical mirror(s) pivoting around at least two axes to deflect the phase-modulated LASER beam 21, and
an exit orifice to send the deflected modulated LASER beam 41 towards the optical focusing system 5.

The optical scanner 4 used is for example a scanning head IntelliScan III from the company SCANLAB AG. The entrance and exit orifices of such an optical scanner 4 have a diameter on the order of 10 to 20 millimeters, and the achievable scanning speeds are on the order of 1 m/s to 10 m/s depending on the focal length of the optics used.

The mirror(s) is/are connected to one (or more) motor(s) to allow their pivoting. This/these motor(s) for the pivoting of the mirror(s) is/are advantageously piloted by the unit of the control unit 6 which will be described in more detail below.

The control unit 6 is programmed to pilot the optical scanner 4 so as to move the pattern 8 along a movement path 42 contained in the focusing plane 71. In some embodiments, the movement path 42 comprises a plurality of cutting segments 42a-42c. The movement path 42 can advantageously have a slot or spiral shape, etc.

The scanning of the beam is of great importance for the result of the obtained cutout. Indeed, the scanning speed used as well as the scanning pitch, are parameters influencing the quality of the cutout.

The use of an optical coupler including an optical fiber 31 of the hollow-body crystal type (rather than an optical assembly composed of mirrors in order to guide the LASER beam 11) makes it possible, when using a multipoint shaping 81, to improve the homogeneity of the energy distribution between the points in the borderline case of very close impact points (center-to-center spacing between two shaped points smaller than the diameter of a point).

In one embodiment, the cutting apparatus further comprises a Dove prism. This is advantageously positioned between the optical color 3 and the optical scanner 4. The Dove prism allows implementing a rotation of the pattern 8 which can be useful in some applications or to limit the size of the area of initiation of each cutting segment 42a-42c.

Advantageously, the control unit 6 can be programmed to activate the femtosecond laser 1 when the scanning speed of the optical scanner 4 is greater than a threshold value. This allows synchronizing the emission of the LASER beam 11 with the scanning of the optical scanner 4. More specifically, the control unit 6 activates the femtosecond laser 1 when the pivoting speed of the mirror(s) of the optical scanner 4 is constant. This allows improving the cutting quality by carrying out a homogeneous surfacing of the cutting plane.

### 2.4. Optical focusing System

The optical focusing system 5 allows moving the focusing plane 71 of the modulated and deflected LASER beam 41 in a cutting plane of the tissue 7 desired by the user.

The optical focusing system 5 comprises:
an entrance orifice to receive the phase-modulated and deflected LASER beam derived from the optical scanner 4,
one (or more) motorized lens(es) to allow its/their movement in translation along the optical path of the phase-modulated and deflected LASER beam, and
an exit orifice to send the focused LASER beam towards the tissue to be treated.

The lens(es) used with the optical focusing system 5 can be flat-field lenses. The flat-field lenses allow obtaining a focusing plane over the entire field XY, unlike the standard lenses for which it is concave. This allows ensuring a constant focused-beam size over the entire field.

The control unit 6 is programmed to pilot the movement of the lens(es) of the optical focusing system 5 along an optical path of the LASER beam so as to move the focusing plane 71 into at least three respective cutting planes 72a-72e so as to form a stack of cutting planes 7 of the tissue. This allows performing a cutout in a volume 74, for example within the context of a refractive surgery.

The control unit 6 is able to pilot the movement of the optical focusing system 5 to move the focusing plane 71 between a first extreme position 72a and a second extreme position 72e, in this order. Advantageously, the second extreme position 72e is closer to the femtosecond laser 1 than the first extreme position 72a.

Thus, the cutting planes 72a-72e are formed by starting with the deepest cutting plane 72a in the tissue and by stacking the successive cutting planes up to the most superficial cutting plane 72e in the tissue 7. Thereby, this avoids the problems associated with the penetration of the LASER beam into the tissue 7. Indeed, the gas bubbles form an Opaque Bubble Layer (known as OBL) preventing the propagation of the energy derived from the LASER beam under them. It is therefore preferable to start by generating the deepest gas bubbles first in order to improve the effectiveness of the cutting apparatus.

Advantageously, the use of an optical coupler including an optical fiber 31 of the hollow-core photonic-crystal type (rather than an optical assembly composed of mirrors in order to guide the LASER beam 11) allows filtering the LASER signal 11 derived from the femtosecond laser by removing its possible aberrations. It is thus possible to reduce the distance between two successive cutting planes (distance between successive cutting planes smaller than the diameter of an impact point) to achieve a high-accuracy cutout in a volume 74.

### 2.5. Control Unit

As indicated above, the control unit 6 allows monitoring the various elements constituting the cutting apparatus, namely the femtosecond laser 1, the shaping system 2, the optical scanner 4 and the optical focusing system 5.

The control unit 6 is connected to these various elements by means of one (or more) communication bus(es) allowing:
the transmission of control signals such as:
   the phase mask to the shaping system,
   the activation signal to the femtosecond laser and the power setpoints,
   the scanning speed to the optical scanner,
   the position of the optical scanner along the movement path,
   the cutting depth to the optical focusing system.
the receipt of measurement data derived from the various elements of the system such as:
   the scanning speed reached by the optical scanner, or
   the position of the optical focusing system, etc.

The control unit 6 can be composed of one (or more) workstation(s), and/or one (or more) computer(s) or can be of any other type known to those skilled in the art. The control unit 6 can for example comprise a mobile phone, an electronic tablet (such as an IPAD®), a Personal Digital Assistant (or “PDA”,), etc. In all cases, the control unit 6 comprises a processor programmed to allow the piloting of the femtosecond laser 1, of the shaping system 2, of the optical scanner 4, of the optical focusing system 5, etc.

### 2.6. Articulated Arm

Thanks to the use of an optical coupler (3) including a photonic-crystal optical fiber (31), the cutting apparatus described above can be mounted in a therapy apparatus including an articulated arm 200 as illustrated in FIG. 6.

The arm 200 comprises several arm segments 201-204 connected by motorized articulations 205-207 (pivot or ball-joint connections) to allow the automatic movement in rotation of the different segments 201-204 relative to each other. Particularly, the arm is articulated to allow the movement of the free end of the arm along three orthogonal axes X, Y and Z:
the axis X, defining a horizontal longitudinal direction,
the axis Y, defining a horizontal transverse direction, which with the axis X defines a horizontal plane XY,
the axis Z, defining a vertical direction, perpendicular to the horizontal plane XY.

The free end of the arm 2 may include an immobilization member equipped with a suction ring capable of suctioning an ocular tissue to be treated and holding it firmly in position.

The arm 2 is for example a TX260L marketed by the company STAUBLI. Advantageously, the shaping system 2, the optical scanner 4 and the optical focusing system 5 can be mounted in the end segment 204 of the arm 200, while the femtosecond laser 1 can be integrated into a movable box 210 of the therapy apparatus, the optical coupler 3 extending between the box 210 and the end segment 204 to propagate the LASER beam 11 derived from the femtosecond laser 1 towards the shaping system 2.

### 3. Conclusions

Thus, the invention allows disposing an effective and accurate cutting tool. The reconfigurable modulation of the wave front of the LASER beam allows generating multiple simultaneous impact points 81 each having a size and a monitored position in the focusing plane 71. These different impact points 81 form a pattern 8 in the focal plane 71 of the modulated LASER beam.

The use of an optical coupler 3 including a hollow-core 311 photonic-crystal fiber 31 allows reducing the distance between the different impact points forming the pattern. Indeed, by limiting the spreading phenomenon of the light spectrum, the optical coupler including a hollow-core photonic-crystal fiber allows making the phase-modulated LASER beam cleaner.

The reader will understand that many modifications can be made to the invention described above without physically departing from the new teachings and advantages described here. Therefore, all modifications of this type are intended to be incorporated within the scope of the appended claims.

1. An apparatus for cutting a tissue, said apparatus including:
a femtosecond laser which emits an initial LASER beam in the form of pulses,
a shaping system including a Spatial Light Modulator (SLM) positioned downstream of the femtosecond laser, wherein said shaping system transforms the initial LASER beam into a unique phase-modulated LASER beam by modulating the phase of the wave front of the initial LASER beam according to a modulation instruction calculated to distribute the energy of the unique phase-modulated LASER beam into at least two peaks of intensity spatially separated in a focusing plane, wherein said at least two peaks of intensity induce the simultaneous production of at least two impact points forming a pattern in a focusing plane, and wherein each impact point generates a cavitation bubble into the tissue,
an optical scanner, positioned downstream of the shaping system, to move the pattern along a predefined movement path in the focusing plane,
an optical focusing system, positioned downstream of the optical scanner, to move the focusing plane of the modulated LASER beam in a desired cutting plane of the tissue, and
a control unit that allows piloting the shaping system, the optical scanner and the optical focusing system,
wherein the apparatus further comprises an optical coupler between the femtosecond laser and the shaping system, the optical coupler including:
a hollow-core photonic-crystal optical fiber which filters the initial LASER beam derived from the femtosecond laser, said hollow-core photonic-crystal fiber including a hollow core placed under vacuum by the vacuum pump and at least one sheath surrounding the hollow core,
a first connection cell for linking the optical coupler to the femtosecond laser, said first connection cell having at least one connection terminal opening out towards the outside of the first connection cell, wherein the at least one connection terminal of the first connection cell is connected to the vacuum pump, and
a second connection cell for linking the optical coupler to the shaping system, said second connection cell having at least one connection terminal opening out towards the outside of the second connection cell, wherein the at least one connection terminal of the second connection cell is connected to the vacuum pump,
and wherein the control unit is configured to activate the vacuum pump in order to place the hollow core under vacuum by sucking gases contained in the hollow core of the photonic-crystal optical fiber via the first and second connection cells located at both ends of the optical fiber.

2. The cutting apparatus according to claim 1, wherein each connection cell is sealingly mounted at a respective end of the photonic-crystal fiber.

3. The cutting apparatus according to claim 1, wherein each connection cell comprises:
an outer shell,
a transmission channel housed in the shell, wherein the transmission channel allows the passage of the LASER beam inside the outer shell, and
a window transparent to the LASER radiation at one end of the transmission channel, wherein the window faces the femtosecond laser or the shaping system.

4. The cutting apparatus according to claim 1, wherein the control unit receives a scanning speed reached by the optical scanner and a position of the optical focusing system.

5. The cutting apparatus according to claim 4, wherein the control unit is programmed to activate the femtosecond laser after determining that the scanning speed is greater than a pre-determined threshold value.

6. The cutting apparatus according to claim 1, wherein the control unit transmits a phase mask to the shaping system.

7. The cutting apparatus according to claim 1, wherein the control unit transmits, to the femtosecond laser, an activation signal and power setpoints.

8. The cutting apparatus according to claim 1, wherein the control unit transmits, to the optical scanner, a scanning speed.

9. The cutting apparatus according to claim 1, wherein the control unit transmits, to the optical scanner, a position on a predefined movement path.

10. The cutting apparatus according to claim 1, wherein the control unit transmits, to the optical focusing system, a cutting depth.

11. A method for cutting a tissue, said method including:
emitting, by a femtosecond laser driven by a control unit, an initial LASER beam in the form of pulses,
transforming, by a shaping system driven by the control unit and including a Spatial Light Modulator (SLM) positioned downstream of the femtosecond laser, the initial LASER beam into a unique phase-modulated LASER beam by modulating the phase of the wave front of the initial LASER beam according to a modulation instruction calculated to distribute the energy of the unique phase-modulated LASER beam into at least two peaks of intensity spatially separated in a focusing plane, wherein said at least two peaks of intensity induce the simultaneous production of at least two impact points forming a pattern in the focusing plane, and wherein each impact point generates a cavitation bubble into the tissue,
moving, by an optical scanner driven by the control unit and positioned downstream of the shaping system, the pattern along a predefined movement path in the focusing plane,
moving, by an optical focusing system driven by the control unit and positioned downstream of the optical scanner, the focusing plane of the modulated LASER beam in a desired cutting plane of the tissue, and
sucking, by a vacuum pump driven by the control unit, gases contained in a hollow core of a photonic-crystal optical fiber having at least one sheath surrounding the hollow core, via first and second connection cells located at both ends of the optical fiber wherein:
the first connection cell links the photonic-crystal optical fiber to the femtosecond laser, and has at least one connection terminal connected to the vacuum pump,
the second connection cell links the photonic-crystal optical fiber to the shaping system, and has at least one connection terminal connected to the vacuum pump,
and wherein said sucking step places the hollow core under vacuum by carrying out a vacuum pumping at each end of the optical fiber.

12. The method according to claim 11, which further comprises the step of:
receiving a scanning speed reached by the optical scanner and a position of the optical focusing system, the step of emitting an initial LASER beam in the form of pulses being carried out if the scanning speed is greater than a pre-determined threshold value.
